(19) United States
(12) Patent Application Publication
(10) Pub. No.: US 2016/0015559 A1
(43) Pub. Date: Jan. 21, 2016

(54) Heated Undergarment for the Treatment of Fibromyalgia

(71) Applicants: Michael Whitsett, Santa Rosa, CA (US); Joan Whitsett, Santa Rosa, CA (US)

(72) Inventors: Michael Whitsett, Santa Rosa, CA (US); Joan Whitsett, Santa Rosa, CA (US)

(21) Appl. No.: 14/335,769

(22) Filed: Jul. 18, 2014

(51) Int. Cl. A61F 7/00

(52) U.S. Cl. CPC A61F 7/007; A61F 2007/0018; A61F 2007/0071; A61F 2007/0078

(57) ABSTRACT

An undergarment for the treatment of Fibromyalgia is provided. The undergarment is shaped to be worn about the torso of a patient and the undergarment includes a plurality of electrical heating elements secured to the undergarment, each of the plurality of electrical heating elements positioned at a location substantially coincident with a Fibromyalgia trigger point of the patient. The use of the undergarment allows the patient to be active while continually receiving therapeutic treatment for the discomfort caused by Fibromyalgia.

### TECHNICAL FIELD

The present application relates generally to an undergarment for the treatment of Fibromyalgia, and more particularly to an undergarment to provide heat to identified Fibromyalgia trigger points for the relief of Fibromyalgia symptoms.

### BACKGROUND

Fibromyalgia is a common disorder which affects the muscles and soft tissue of the body. Symptoms of Fibromyalgia include chronic muscle pain, fatigue, difficulty sleeping and painful tender points or trigger points on the body.

Fibromyalgia is commonly diagnosed through the palpation of the location of one or more Fibromyalgia trigger points on the body of a patient. The Fibromyalgia trigger points are localized areas of tenderness under the surface of the skin of the patient and are generally located over the neck, back, chest, elbows, hips, buttocks and knees. Research has identified eighteen Fibromyalgia trigger points on the body. Pain as a result of pressure to eleven or more of these eighteen trigger points commonly results in a diagnosis of Fibromyalgia.

Over-the-counter and prescription pain relievers, antidepressants and anti-seizure medications are commonly prescribed for the treatment of the symptoms of Fibromyalgia with the goal of reducing the pain caused by the disorder. However, due to the possible side-effects of the prescribed medications and a general adversity to medications, many patients may prefer a drug-free option for the manageability of the pain caused by Fibromyalgia.

Accordingly, there is a need in the art for a system and method for drug-free manageability of the pain caused by Fibromyalgia.

### SUMMARY

A therapeutic system and method for the treatment of pain associated with Fibromyalgia syndrome is described.

In accordance with an embodiment of the present invention, an undergarment for the treatment of Fibromyalgia is provided. The undergarment is shaped to be worn about the torso of a patient and includes a plurality of electrical heating elements secured to the undergarment, each of the plurality of electrical heating elements is positioned at a location substantially coincident with a Fibromyalgia trigger point of the patient. In one embodiment, the electrical heating elements are grouped into one or more treatment zones which are individually addressable by the patient to provide a desired therapeutic effect for the treatment of the painful trigger points associated with Fibromyalgia syndrome.

The present invention additionally provides a method for the treatment of Fibromyalgia which includes, positioning an undergarment on the torso of a patient and applying therapeutically sufficient heat through a plurality of electrical heating elements secured to the undergarment, each of the plurality of electrical heating elements positioned at a location substantially coincident with one or more Fibromyalgia triggers point of the patient.

### BRIEF DESCRIPTION OF THE DRAWINGS

FIG. 1A is a diagrammatic view of the Fibromyalgia trigger points located on the front side of the torso of a patient suffering from Fibromyalgia syndrome.

FIG. 1B is a diagrammatic view of the Fibromyalgia trigger points located on the back side of the torso of a patient suffering from Fibromyalgia syndrome.

FIG. 2A is a diagrammatic view of the front side of an undergarment for the treatment of Fibromyalgia in accordance with an embodiment of the present invention.

FIG. 2B is a diagrammatic view of the back side of an upper torso undergarment for the treatment of Fibromyalgia in accordance with an embodiment of the present invention.

FIG. 3A is diagrammatic view of the front side of an upper torso undergarment for the treatment of Fibromyalgia in accordance with an embodiment of the present invention.

FIG. 3B is a diagrammatic view of the back side of an upper torso undergarment for the treatment of Fibromyalgia in accordance with an embodiment of the present invention.

FIG. 4 is a diagrammatic view of the back side of a lower torso undergarment for the treatment of Fibromyalgia in accordance with an embodiment of the present invention.

### DETAILED DESCRIPTION

Those of ordinary skill in the art will realize that the following detailed description of embodiments in this specification is illustrative only, and is not intended to be in any way limiting. Other embodiments will readily suggest themselves to such skilled persons having the benefit of this disclosure. It will be apparent to one skilled in the art that these specific details may not be required to practice the embodiments. In the following description of the embodiments, substantially the same parts are denoted by the same reference numerals.

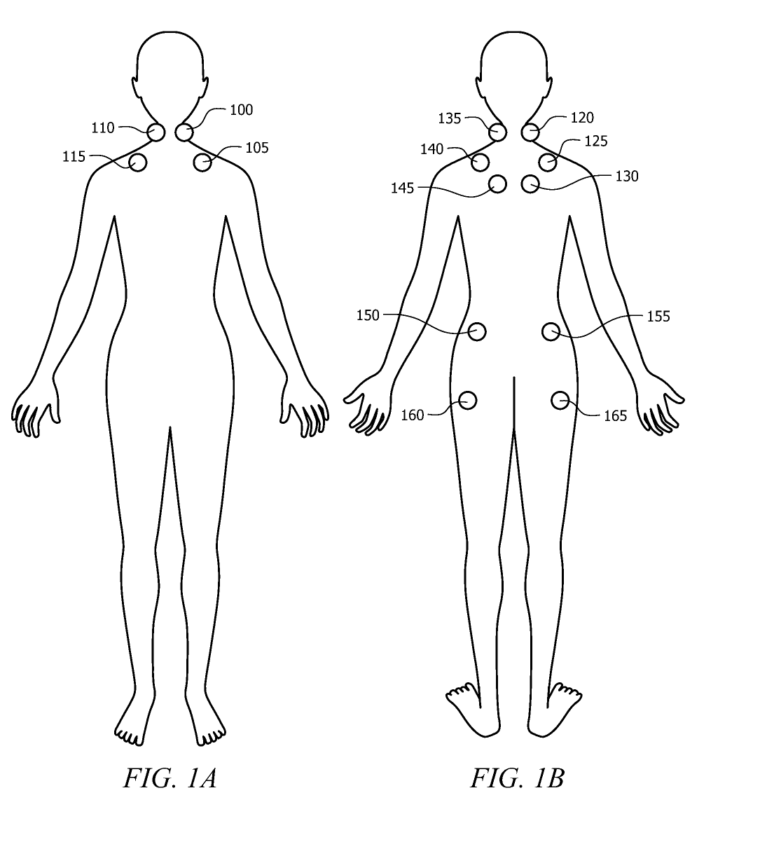

With reference to FIG. 1A and FIG. 1B, patients suffering from Fibromyalgia frequently experience tenderness at one or more of eighteen specific locations on the body, commonly referred to as Fibromyalgia trigger points. Fourteen of the identified Fibromyalgia trigger points are located on the torso. In the present invention, the torso is an anatomical term for the central part of the human body, exclusive of the head and limbs. As shown with reference to FIG. 1A, four of the trigger points 100, 105, 110, 115 are located on the front side of the torso, within the upper portion of the torso. Additionally, as shown with reference to FIG. 1B, ten of the trigger points are located on the back side of the torso, four within the upper back 120, 125, 135, 140, two within the mid-back 130, 145, two within the lower back or hips 150, 155 and two at the lower buttocks 160, 165. While the exact location of the trigger points may vary from patient to patient, research has shown that the general areas of the trigger points located on the torso of the patient are consistent with a diagnosis of Fibromyalgia syndrome. Patients suffering from Fibromyalgia often find relief from discomfort through the application of heat to these identified trigger points. However, the typical means of applying heat to the trigger points requires that the patient remain sedentary, such as when placing a heating pad over the trigger point. Additionally, once the heat is removed from the trigger point, the patient's discomfort often returns.

In the present invention, an undergarment for the treatment of Fibromyalgia is provided.

The undergarment is configured to surround the torso of a patient and to provide a controllable source of heat to the Fibromyalgia trigger points. The use of the undergarment allows the patient to be active while continually receiving therapeutic treatment for the discomfort caused by Fibromyalgia.

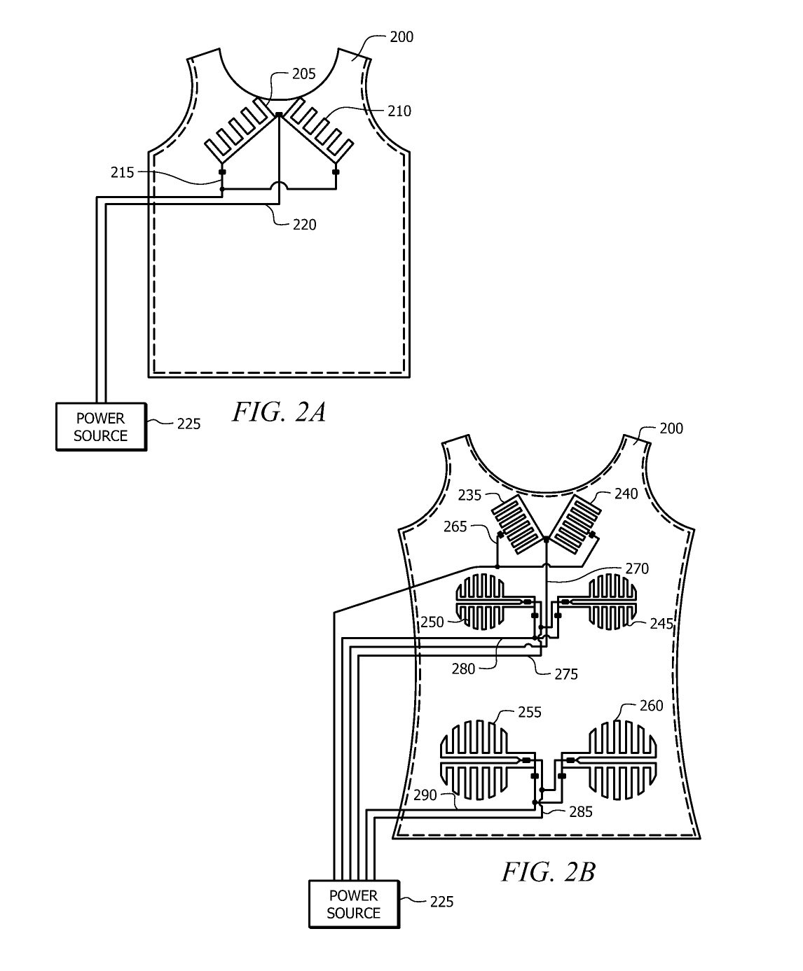

With reference to FIG. 2A and FIG. 2B, in one embodiment of the present invention, an undergarment 200 for the treatment of Fibromyalgia is shaped to be worn about the torso of a patient and the undergarment 200 includes a plurality of electrical heating elements secured to the undergarment, each of the plurality of electrical heating elements positioned at a location substantially coincident with a Fibromyalgia trigger point of the patient.

FIG. 2A illustrates a front view of the undergarment 200 having two electrical heating elements 205, 210 positioned at the location of four of the Fibromyalgia trigger points 100, 105, 110, 115, on the upper front torso of the patient. The electrical heating elements 205, 210 are coupled to a power source 225 through a first electrical conductor 215 and a second electrical conductor 220. The first electrical conductor 215 may be coupled to the positive terminal of the power source 225 and the second electrical conductor 220 may be coupled to the negative terminal of the power source 225, as is commonly known for the application of power to an electrical element.

FIG. 2B illustrates a back view of the undergarment 200 having six electrical heating elements 235, 240, 245, 250, 255, 260. Two of the six electrical heating elements 235, 240 are positioned at the location of four of the Fibromyalgia trigger points 120, 125, 135, 140 on the upper back of the patient. Two of the six electrical heating elements 245, 250 are positioned at the location of two of the Fibromyalgia trigger points 130, 145 on the mid-back of the patient. Two of the six electrical heating elements 255, 260 are positioned at the location of two of the Fibromyalgia trigger points 150, 155 on the lower back and buttocks of the patient. The electrical heating elements 235, 240 are coupled to a power source 225 through a first electrical conductor 265 and a second electrical conductor 270. The electrical heating elements 245, 250 are coupled to a power source 225 through a first electrical conductor 280 and a second electrical conductor 275. The electrical heating elements 255, 260 are coupled to a power source 225 through a first electrical conductor 265 and a second electrical conductor 270. The first electrical conductor of each of the electrical heating elements may be coupled to the positive terminal of the power source and the second electrical conductor of the electrical heating elements may be coupled to the negative terminal of the power source 225, as is commonly known for the application of power to an electrical element.

In one embodiment, the power source may be a battery coupled to each of the electrical heating elements. In a particular embodiment, the battery may be a rechargeable battery, such as a lithium ion battery. Additionally, the power source may be an AC to DC voltage converter which can provide power to the electrical heating elements through a standard AC supply, such as a wall outlet.

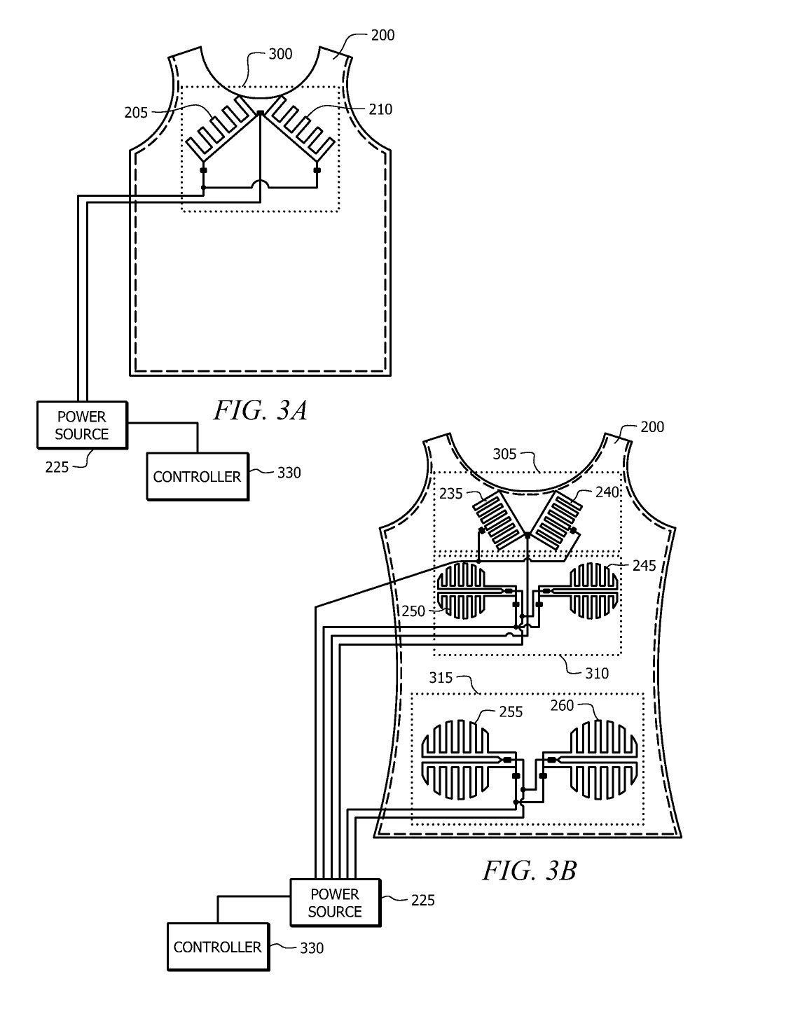

With reference to FIG. 3A and FIG. 3B, the undergarment may further include a controller 330 coupled to the power source 225 for individually activating and deactivating one or more of the electrical heating elements on the front side of the undergarment 205, 210 and one or more of the electrical heating elements on the back side of the undergarment 235, 240, 245, 250, 255, 260. The controller 300 may be programmed to automatically provide therapeutic heating to one or more of the trigger points through one or more of the electrical heating elements 205, 210,235, 240, 245, 250, 255, 260. Alternatively, the controller 300 may be controllable by the patient to provide therapeutic heating to one or more of the trigger points through one or more of the electrical heating elements 205, 210, 235, 240, 245, 250, 255, 260 as desired by the patient. The controller 300 may also be used to regulate the power provided by the power source to one or more of the electrical heating element 205, 210, 235, 240, 245, 250, 255, 260, thereby controlling the temperature of the heat generated by the electrical heating element.

In the embodiment illustrated with reference to FIG. 3A and FIG. 3B, each of the electrical heating elements is grouped into an individually addressable treatment zone. Electrical heating elements 205, 210 are assigned to a first individually addressable treatment zone 300 located on the front side of the undergarment 200. Electrical heating elements 235, 240 are assigned to a second individually addressable treatment zone 305 located on the upper back side of the undergarment 200. Electrical heating elements 245, 250 are assigned to a third individually addressable treatment zone 310 located on the mid-back side of the undergarment 200 and electrical heating elements 355, 260 are assigned to a fourth individually addressable treatment zone 315 located on the lower back side of the undergarment 200. The controller 330 is configured to provide power from the power source 225 individually to the heating elements in each of the treatment zones 300, 305, 310, 315 and to regulate the amount of power provided to each of the treatment zones. As such, the controller can be configured automatically, or by the patient, to provide heating at a first temperature to the first treatment zone 300 and to provide heat at a second temperature to the second treatment zone 305, wherein the first temperature is different than the second temperature. The illustrated assignment of the heating elements to treatment zones is not meant to be limiting and various other assignments of the electrical heating elements are within the scope of the present invention.

In the specific embodiment illustrated with reference to FIG. 2A and FIG. 2B, the undergarment 200 is characterized as a sleeveless undershirt to be worn underneath clothing and in direct contact with the skin of a patient. It is additionally within the scope of the present invention for the electrical heating elements to be incorporated into other undergarments, such as an undershirt having short sleeves or long sleeves. Additionally, for providing therapeutic heating to the trigger points 150, 155, 160, 165 located on the lower torso of a patient, it is within the scope of the invention to incorporate the electrical heating elements into a brief or underpants to be worn by the patient.

In a particular embodiment, the undergarment 200 is made of a knit fabric material, such as cotton or rayon jersey. The elasticity of the material, the shape and the size of the undergarment 200 are intended to provide a close fit between the undergarment 200 and the patient such that the electrical heating element if the undergarment 200 are positioned close to the skin of the patient. The electrical heating elements 205, 210, 235, 240, 245, 250, 255, 260 are sized to at least surround the Fibromyalgia trigger points of the patient. The electrical heating elements 205, 210, 235, 240, 245, 250, 255, 260 may be fabricated of stainless steel thread, carbon fiber thread or metal-wound synthetic fiber. In general, the electrical heating elements 205, 210, 235, 240, 245, 250, 255, 260 are fabricated of an electrically conductive material possessing a high enough electrical resistance to generate a desirable level of heat from the power supply 225. The spacing of the electrical conductors, the power supply voltage and the number of electrical heating elements connected in parallel are adjustable to achieve a desired level of heat generation to provide therapeutic temperatures to the trigger points of the patient. In a specific embodiment, approximately 0.24 watts/in² is sufficient to increase the temperature of the skin at the trigger points to about 108 degrees.

The electrical heating elements 205, 210, 235, 240, 245, 250, 255, 260 may be designed in various configurations. As shown in FIG. 2B, electrical heating elements 245, 250, 255, 260 are designed to substantially form a circular element by the positioning of the electrically conductive thread. Additionally, electrical heating elements 235 and 240 are designed to substantially for a comb element through the positioning of the electrically conductive thread. The electrical heating elements 205, 210, 235, 240, 245, 250, 255, 260 attached to the undergarment 200 by stitching the electrically conductive thread to the undergarment 200 using a zig-zag stitch over the heating element, alternatively, the conductive thread of the electrical heating elements 205, 210, 235, 240, 245, 250, 255, 260 may be wound on a sewing machine bobbin and stitched directly to the fabric of the undergarment 200. In an additional embodiment, the electrical heating elements 205, 210, 235, 240, 245, 250, 255, 260 may be attached to the undergarment 200 using a heat-fusible adhesive on a stretchable backing, such as heat fusible knit interfacing. It is desirable that the method of securing the electrical heating elements 205, 210, 235, 240, 245, 250, 255, 260 to the undergarment 200 maintain the elasticity of the fabric, in at least the circumferential direction, to allow the undergarment 200 to conform closely to the body of the patient and to efficiently transfer heat from the electrical heating elements 205, 210, 235, 240, 245, 250, 255, 260 to the trigger points. The electrical conductors coupling the electrical heating elements 205, 210, 235, 240, 245, 250, 255, 260 to the power source may be encapsulated in a non-conductive, waterproof, polymer to ensure the long-term integrity of the electrical conductors.

It is additionally within the scope of the invention to provide adjustability of the location of the electrical heating elements 205, 210, 235, 240, 245, 250, 255, 260 on the undergarment 200. It is desirable to be able to adjust the location of the electrical heating elements 205, 210, 235, 240, 245, 250, 255, 260 to accommodate for differences in the specific location of the trigger points on various patients. The electrical heating elements 205, 210, 235, 240, 245, 250, 255, 260 may be prefabricated and may be removably attached to the undergarment 200 using various techniques known in the art, including, but not limited to the use of hook-and-loop fasteners.

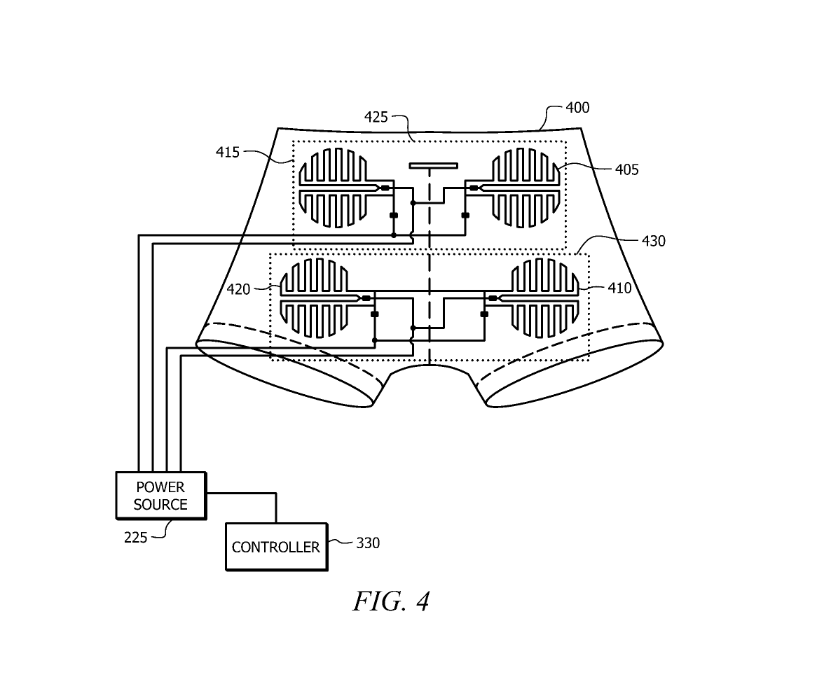

With reference to FIG. 4, in an additional embodiment, a brief or underpants 400 designed to be worn on the lower torso of the patient may additionally be provided for delivering therapeutic heating to the trigger points 150, 155, 160, 165 located on the lower torso of a patient. FIG. 4 illustrates a back view of the lower torso undergarment 400 having two electrical heating elements 405, 415 positioned at Fibromyalgia trigger points 150, 155, which are located at the lower back or hips of the patient and two electrical heating elements 410, 420 positioned at Fibromyalgia trigger points 160, 165, which are located at the lower buttocks of the patient. The electrical heating elements 405, 410, 415, 420 are coupled to a power source 225 as previously described with reference to FIG. 2. With reference to FIG. 4, the lower torso undergarment may further include a controller 330 coupled to the power source 225 for individually activating and deactivating one or more of the electrical heating elements on the back side of the undergarment 405, 410, 415, 420 as previously described with reference to FIG. 3.

In the embodiment illustrated with reference to FIG. 4, each of the electrical heating elements is grouped into an individually addressable treatment zone. Electrical heating elements 405, 415 are assigned to a first individually addressable treatment zone 425 on the undergarment 400. Electrical heating elements 410, 420 are assigned to a second individually addressable treatment zone 430 located on the undergarment 400. The controller 330 is configured to provide power from the power source 225 individually to the heating elements in each of the treatment zones 425, 430 and to regulate the amount of power provided to each of the treatment zones as previously described with reference to FIG. 3.

In operation, the undergarment 200, 400 may be positioned on the torso of a patient and the power supply may be controlled to apply therapeutically sufficient heat to one or more Fibromyalgia trigger points of a patient through a plurality of electrical heating elements 205, 210, 235, 240, 245, 250, 255, 260, 405, 410, 415, 420 secured to the undergarment, each of the plurality of electrical heating elements 205, 210, 235, 240, 245, 250, 255, 260, 405, 410, 415, 420 being positioned at a location substantially coincident with a Fibromyalgia trigger point of the patient. The Fibromyalgia undergarment 200, 400 provides therapeutic treatment for the discomfort associated with Fibromyalgia without the use of mediations and without negatively affecting the mobility of the patient.

The foregoing descriptions of specific embodiments of have been presented for purposes of illustration and description. They are not intended to be exhaustive or to limit the invention to the precise forms disclosed, and obviously many modifications and variations are possible in light of the above teaching. The embodiments were chosen and described in order to best explain the principles and practical applications, to thereby enable others skilled in the art to best utilize the various embodiments with various modifications as are suited to the particular use contemplated. It is intended that the scope be defined by the claims appended hereto and their equivalents.

1. An undergarment for the treatment of Fibromyalgia, the undergarment comprising:
an undergarment shaped to be worn about the torso of a patient; and
a plurality of electrical heating elements secured to the undergarment, each of the plurality of electrical heating elements positioned at a location substantially coincident with a Fibromyalgia trigger point of the patient.

2. The undergarment of claim 1, further comprising an electrical conductor coupled to each of the plurality of electrical heating elements.

3. The undergarment of claim 3, further comprising a power source coupled to each of the electrical conductors coupled to each of the plurality of electrical heating elements.

4. The undergarment of claim 3, wherein the power source is a battery.

5. The undergarment of claim 3, wherein the power source is a rechargeable battery.

6. The undergarment of claim 3, wherein the power source is AC to DC voltage converter.

7. The undergarment of claim 3, further comprising a controller configured for controlling the power source to provide power to one or more of the plurality of electrical heating elements.

8. The undergarment of claim 1, wherein each of the plurality of electrical heating elements belongs to one of a plurality of individually addressable treatment zones.

9. The undergarment of claim 8, further comprising a controller configured for controlling the power source to provide power to each of the electrical heating elements of one or more of the individually addressable treatment zones.

10. The undergarment of claim 1, wherein each of the plurality of electrical heating elements are configured to generate sufficient heat to achieve therapeutic treatment of the Fibromyalgia trigger point.

11. The undergarment of claim 1, wherein each of the plurality of electrical heating elements are dimensioned to sufficiently surround the Fibromyalgia trigger point of the patient.

12. The undergarment of claim 1, wherein the location of each of the plurality of electrical heating elements is adjustable within a range of the Fibromyalgia trigger point of the patient.

13. The undergarment of claim 1, wherein each of the pluralities of electrical heating elements are electrically conductive and sufficiently electrically resistant to generate a therapeutic heat when coupled to a power source.

14. The undergarment of claim 1, wherein each of the pluralities of electrical heating elements are comprised of an electrically conductive materials selected from the electrically conductive materials consisting of stainless steel thread, carbon fiber thread and metal-wound synthetic fiber.

15. The undergarment of claim 1, wherein the undergarment is comprised of a knit fabric.

16. The undergarment of claim 1, wherein the electrical heating elements are sewn into the undergarment.

17. An undergarment for the treatment of Fibromyalgia, the undergarment comprising:
an undergarment shaped to be worn about the torso of a patient;
a plurality of electrical heating elements secured to the undergarment, each of the plurality of electrical heating elements positioned at a location substantially coincident with a Fibromyalgia trigger point of the patient, and each of the plurality of electrical heating elements belonging to one of a plurality of individually addressable treatment zones;
an electrical conductor coupled to each of the plurality of electrical heating elements;
a removable power source coupled to each of the plurality of electrical conductors; and
a controller coupled to the power source, the controller configured for controlling the power source to provide power to one or more of the individually addressable treatment zones.

18. The undergarment of claim 17, wherein the power source is a battery.

19. The undergarment of claim 17, wherein the power source is an AC to DC voltage converter.

20. A method for the treatment of Fibromyalgia, the method comprising:
positioning an undergarment on the torso of a patient; and
applying therapeutically sufficient heat through a plurality of electrical heating elements secured to the undergarment, each of the plurality of electrical heating elements positioned at a location substantially coincident with one or more Fibromyalgia trigger points of the patient.
